(19) United States
(12) Patent Application Publication
(10) Pub. No.: US 2024/0324985 A1
(43) Pub. Date: Oct. 3, 2024

(54) Multimodal Imaging Means for Evaluating Tissue Oxygenation Status, Its Working Method and Application

(71) Applicant: Beijing Union Medical College Hospital of the Chinese Academy of Medical Sciences, Beijing (CN)

(72) Inventors: Meng YANG, Beijing (CN); Rui ZHANG, Beijing (CN); Sirui LIU, Beijing (CN); Yixiu ZHANG, Beijing (CN); Ming WANG, Beijing (CN); Ruina ZHAO, Beijing (CN); Yuxin JIANG, Beijing (CN)

(73) Assignee: Beijing Union Medical College Hospital of the Chinese Academy of Medical Sciences, Beijing (CN)

(21) Appl. No.: 18/624,275

(22) Filed: Apr. 2, 2024

(30) Foreign Application Priority Data
Apr. 3, 2023 (CN) 2023103420225

(51) Int. Cl. A61B 8/08; A61B 5/00; A61B 5/0205; A61B 5/145

(52) U.S. Cl. CPC A61B 8/0866; A61B 5/0095; A61B 5/0205; A61B 5/14542; A61B 8/5261; A61B 2503/40; A61B 2503/42

(57) ABSTRACT

A multimodal imaging means for evaluating tissue oxygenation status, its working method and application are provided. The means includes: a test object acquisition module, a basic data measurement module, a grayscale ultrasound module, a photoacoustic imaging module, a planar wave imaging module, a calculation module, and a statistical analysis module. Structural information, oxygenation status data, and vascular density information of placenta of pregnant rats are obtained, diagnostic ability AUC of receiver operating characteristics ROC in a diagnostic mode is calculated with a standard method, and statistical analysis is conducted. It can evaluate tissue oxygen saturation in real-time and non-invasive manner, and an evaluation result is more accurate.

### CROSS-REFERENCE TO THE RELATED APPLICATIONS

This application is based upon and claims priority to Chinese Patent Application No. CN 2023103420225, filed on Apr. 3, 2023, the entire contents of which are incorporated herein by reference.

### TECHNICAL FIELD

The present invention relates to the technical field of medical image processing, particularly to a multimodal imaging means for evaluating tissue oxygenation status, as well as its working method and application of the means, which is mainly used for processing images of grayscale ultrasound imaging, photoacoustic imaging, and plane wave imaging.

### BACKGROUND

Preeclampsia is one of the most common complications of pregnancy, which can rapidly develop into serious consequences such as maternal and fetal death. At present, a diagnosis of preeclampsia depends on clinical manifestations, including hypertension and proteinuria at 20 weeks of pregnancy and/or less than 48 hours after delivery. However, once hypertension occurs, preeclampsia can quickly worsen into a life-threatening hypertensive crisis for pregnant women. Due to lack of a timely and accurate diagnosis of clinical manifestations, there is an urgent need to develop more accurate, sensitive, and non-invasive diagnostic indicators for preeclampsia.

Photoacoustic imaging is an emerging imaging technology that can image common endogenous chromophores, including water, oxygenated hemoglobin (HbO₂), deoxyhemoglobin (Hb), melanin, and lipid. Due to its ultrasound and optical properties, this method can simultaneously evaluate chemical composition and structural characteristics of tissues, and has advantages such as high resolution and non-invasive. Photoacoustic imaging, with its strength, has developed rapidly in recent years and has been widely used in fields such as brain, thyroid, breast, skin, lymphatic system, gynecology, urinary system imaging, and intraoperative imaging. An ability of photoacoustic imaging to evaluate tissue oxygenation status has been confirmed. In addition, an oxygenation capacity of a placenta is not only determined by its oxygenation status, but also closely related to its neovascularization status. Therefore, information on placental vascularization will help further improve an accuracy of diagnosis.

Plane wave imaging is a microvascular blood flow imaging method based on non focusing wave imaging technology. Thanks to a fast processing platform and effective wall filtering algorithms, plane wave imaging distinguishes blood flow signals from low-speed tissue movement and displays an ability to display microvascular morphology. Compared with power Doppler, plane wave imaging can still more clearly display small blood vessels in the placenta at 32 weeks of pregnancy, even in the presence of respiratory movement in a pregnant. Therefore, plane wave imaging has advantages in early diagnosis of placental neovascularization. In preeclampsia, an invasion of a superficial trophoblast layer leads to incomplete remodeling of maternal spiral blood vessels, resulting in high resistance, low volume blood vessels, and pathological changes in vascular smooth muscle tissue that affect placental angiogenesis. Therefore, using plane wave imaging to evaluate placental blood perfusion and distribution will help in an early diagnosis of preeclampsia.

Although the above imaging methods can provide real-time and non-invasive important information on the placenta, a single imaging method can only provide limited structural or functional information at present and cannot provide a more comprehensive evaluation of the placenta. A multimodal imaging method that integrates multiple imaging techniques such as grayscale ultrasound, photoacoustic imaging, and plane wave imaging can provide morphological and functional information simultaneously.

### SUMMARY

In order to overcome the defects of the prior art, the technical problem to be solved by the invention is to provide a multimodal imaging means for evaluating tissue oxygenation status, which can combine traditional ultrasound grayscale imaging, photoacoustic imaging, and plane wave imaging to perform multi parameter imaging on the structure and function of the placenta, enabling real-time and non-invasive evaluation of tissue oxygenation status, achieving in a more accurate evaluation result.

The technical scheme of the invention is as follows. The multimodal imaging means for evaluating tissue oxygenation status, comprises:
a test object acquisition module, which is configured to randomly divide pregnant rats into a hypoxia group and a normoxia group, with an oxygen concentration of 12+2% in the hypoxia group and 20+2% in the normoxia group,
a basic data measurement module, which is configured to measure blood pressure, proteinuria, and fetal mass on the 18th day of pregnancy,
a grayscale ultrasound module, which is configured to obtain placental structural information provided by grayscale ultrasound on the 18th day of pregnancy,
a photoacoustic imaging module, which is configured to obtain oxygenation status data of the rat placenta on the 18th day of pregnancy,
a planar wave imaging module, which is configured to perform imaging on a transverse plane of an umbilical cord placental inlet of each placenta in the rats on the 18th day of pregnancy to obtain vascular density,
a calculation module, which is configured to calculate a diagnostic capability AUC of a receiver operating characteristics ROC in a diagnostic mode with a standard method,
a statistical analysis module, which is configured to perform statistical analysis on data of the grayscale ultrasound module, the photoacoustic imaging module, and the plane wave imaging module.

In the present invention structural information, oxygenation status data, and vascular density information of placenta of pregnant rats are obtained, diagnostic ability AUC of receiver operating characteristics ROC in a diagnostic mode is calculated with a standard method, and statistical analysis is conducted. It can evaluate tissue oxygen saturation in real-time and non-invasive manner, and an evaluation result is more accurate.

A working method for the multimodal imaging means for evaluating tissue oxygenation status is also provided, which comprises the following steps:
(1) dividing pregnant rats into a hypoxia group and a normoxia group randomly, with an oxygen concentration of 12±2% in the hypoxia group and 20±2% in the normoxia group,
(2) measuring blood pressure, proteinuria, and fetal mass on the 18th day of pregnancy,
(3) obtaining placental structural data on the 18th day of pregnancy,
(4) obtaining oxygenation status data of the rat placenta on the 18th day of pregnancy,
(5) performing imaging on the transverse section of the umbilical cord placental inlet of each placenta in the rats to obtain vascular density on the 18th day of pregnancy,
(6) calculating the AUC of the receiver operating characteristics ROC in the diagnostic mode with the standard method,
(7) performing statistical analysis on the data from step (3) to (5), including placental structural information, placental oxygenation status data, and vascular density.

It also provides an application of the multimodal imaging means for evaluating tissue oxygenation status, which is used to diagnose preeclampsia and provide a dynamic monitoring in a treatment of preeclampsia.

One aspect of the present invention is directed to a multimodal imaging means for evaluating tissue oxygenation status. The means includes:
a test object acquisition module, which is configured to randomly divide pregnant rats into a hypoxia group and a normoxia group, with an oxygen concentration of 12±2% in the hypoxia group and 20±2% in the normoxia group;
a basic data measurement module, which is configured to measure blood pressure, proteinuria, and fetal mass on the 18th day of pregnancy;
a grayscale ultrasound module, which is configured to obtain placental structural information provided by grayscale ultrasound on the 18th day of pregnancy;
a photoacoustic imaging module, which is configured to obtain oxygenation status data of the rat placenta on the 18th day of pregnancy;
a planar wave imaging module, which is configured to perform imaging on a transverse plane of an umbilical cord placental inlet of each placenta in the rats on the 18th day of pregnancy to obtain vascular density;
a calculation module, which is configured to calculate a diagnostic capability AUC of a receiver operating characteristics ROC in a diagnostic mode with a standard method; and
a statistical analysis module, which is configured to perform statistical analysis on data of the grayscale ultrasound module, the photoacoustic imaging module, and the planar wave imaging module.

In one embodiment, in the grayscale ultrasound module, a 20 MHz probe is used to identify a reproductive system of a pregnant rat, a cervix of the pregnant rat is displayed in a transverse section of a lower abdomen of the pregnant rat, after displaying clearly, the probe is rotated and adjusted to a direction along a long axis of a left uterine angle, and a first gestational sac appearing along a lower place of the left uterine angle is displayed, a depth and focus of the probe are adjusted to make a fetal rat and placenta inside a target gestational sac clear, the fetal rat and placenta are continuously scanned to observe their morphology and positional relationship, a length of the fetal rat is measured in a sagittal plane and recorded, a complete morphology of the placenta at the umbilical cord placental inlet is displayed, a circumference and area of the placenta are outlined and recorded, subsequently, moving along the left uterine angle, locating the last gestational sac on an upper left side, repeating the above observation and measurement operations, performing the same procedure is performed for a first gestational sac at a lower right corner of a right uterine angle and a last gestational sac at an upper right corner of a right uterine angle, collecting four target gestational sacs from each pregnant rat: lower left, upper left, lower right, and upper right, and taking all measurements three times and taking an average.

In another embodiment, in the photoacoustic imaging module, a guide pad is placed on the abdomen of the pregnant rat, a 9 MHz photoacoustic ultrasound probe is used to display the cervix of the pregnant rat in the transverse section of the lower abdomen, the probe is rotated and adjusted to a direction along a long axis of a left uterine angle, a first gestational sac appearing along a lower place of the left uterine angle is displayed, a depth and focus of the probe is adjusted to make a fetal rat and placenta inside a target gestational sac clear, the probe is switched to a photoacoustic mode in the maximum sagittal section of the placenta, a photoacoustic imaging range is adjusted to include the placenta but not exceed its maximum longitudinal diameter, a distribution of photoacoustic signals is observed, their oxygenation status are calculated and recorded, subsequently, moving along the left uterine angle, locating the last gestational sac on an upper left side, repeating the above observation and measurement operations, performing the same procedure is performed for a first gestational sac at a lower right corner of a right uterine angle and a last gestational sac at an upper right corner of a right uterine angle, collecting four target gestational sacs from each pregnant rat: lower left, upper left, lower right, and upper right, and taking all measurements three times and taking an average.

In another embodiment, in the planar wave imaging module, the cervix of the pregnant rat is displayed in the transverse section of the lower abdomen of the pregnant rat, after the image is clearly displayed, the probe is rotated and adjusted to a direction along a long axis of a left uterine angle, and a first gestational sac appearing along a lower place of the left uterine angle is displayed, a depth and focus of the probe are adjusted to make a fetal rat and placenta inside a target gestational sac clear, the maximum coronal section of the placenta through the umbilical cord placental inlet is displayed, the probe is switched to a plane wave mode, a speed scale is adjusted to 4.5 cm/s, and the gain is set to 50 dB, a fine vascular structure and color signal distribution are observed, after the image is stable, each target placenta is outlined, the area of the placenta in the section and the area of the blood vessels displayed in the plane wave are calculated, the area of the blood vessels in the plane wave is divided by the area of the placenta, and the blood vessel density of the placenta is obtained, subsequently, moving along the left uterine angle, locating the last gestational sac on an upper left side, repeating the above observation and measurement operations, performing the same procedure is performed for a first gestational sac at a lower right corner of a right uterine angle and a last gestational sac at an upper right corner of a right uterine angle, collecting four target gestational sacs from each pregnant rat: lower left, upper left, lower right, and upper right, and taking all measurements three times and taking an average.

In another embodiment, in the calculation module, a diagnostic capability AUC of a receiver operating characteristics ROC in a diagnostic mode is calculated with a standard method.

In another embodiment, in the statistical analysis module, the statistical analysis on the data of the grayscale ultrasound module, the photoacoustic imaging module, and the plane wave imaging module is performed.

Another aspect of the present invention is directed to a working method for the multimodal imaging means for evaluating tissue oxygenation status described above. The method includes the following steps:
(1) dividing pregnant rats into a hypoxia group and a normoxia group randomly, with an oxygen concentration of 12±2% in the hypoxia group and 20±2% in the normoxia group;
(2) measuring blood pressure, proteinuria, and fetal mass on the 18th day of pregnancy;
(3) obtaining placental structural data on the 18th day of pregnancy;
(4) obtaining oxygenation status data of the rat placenta on the 18th day of pregnancy;
(5) performing imaging on the transverse section of the umbilical cord placental inlet of each placenta in rats to obtain vascular density on the 18th day of pregnancy;
(6) calculating the AUC of the receiver operating characteristics ROC in the diagnostic mode with the standard method; and
(7) performing statistical analysis on the data from step (3) to (5), including placental structural information, placental oxygenation status data, and vascular density.

Another aspect of the present invention is directed to an application for the multimodal imaging means for evaluating tissue oxygenation status described above. The application is used to diagnose preeclampsia and provide a dynamic monitoring in a treatment of preeclampsia.

### BRIEF DESCRIPTION OF THE DRAWINGS

The accompanying drawing is used to further illustrate the present disclosure and constitutes a part of the specification. The accompanying drawing, together with the example of the present disclosure, is provided to explain the present disclosure, but does not constitute a limitation to the present disclosure.

The drawing is a flowchart of the multimodal imaging means for evaluating tissue oxygenation status according to the present invention.

### DETAILED DESCRIPTION OF THE EMBODIMENTS

To clarify the technical problems addressed, technical schemes, and the advantages of the present invention, a detailed description is provided below, accompanied by attached drawings and specific embodiments.

The multimodal imaging means for evaluating tissue oxygenation status, comprises:
a test object acquisition module, which is configured to randomly divide pregnant rats into a hypoxia group and a normoxia group, with an oxygen concentration of 12±2% in the hypoxia group and 20±2% in the normoxia group,
a basic data measurement module, which is configured to measure blood pressure, proteinuria, and fetal mass on the 18th day of pregnancy,
a grayscale ultrasound module, which is configured to obtain placental structural information provided by grayscale ultrasound on the 18th day of pregnancy,
a photoacoustic imaging module, which is configured to obtain oxygenation status data of the rat placenta on the 18th day of pregnancy,
a planar wave imaging module, which is configured to perform imaging on a transverse plane of an umbilical cord placental inlet of each placenta in the rats on the 18th day of pregnancy to obtain vascular density,
a calculation module, which is configured to calculate a diagnostic capability AUC of a receiver operating characteristics ROC in a diagnostic mode with a standard method,
a statistical analysis module, which is configured to perform statistical analysis on data of the grayscale ultrasound module, the photoacoustic imaging module, and the plane wave imaging module.

In the present invention structural information, oxygenation status data, and vascular density information of placenta of pregnant rats are obtained, diagnostic ability AUC of receiver operating characteristics ROC in a diagnostic mode is calculated with a standard method, and statistical analysis is conducted. It can evaluate tissue oxygen saturation in real-time and non-invasive manner, and an evaluation result is more accurate.

Preferably, in the grayscale ultrasound module, a 20 MHz probe is used to identify a reproductive system of a pregnant rat. A cervix of the pregnant rat is displayed in a transverse section of a lower abdomen of the pregnant rat. After displaying clearly, the probe is rotated and adjusted to a direction along a long axis of a left uterine angle, and a first gestational sac appearing along a lower place of the left uterine angle is displayed. A depth and focus of the probe are adjusted to make a fetal rat and placenta inside a target gestational sac clear. The fetal rat and placenta are continuously scanned to observe their morphology and positional relationship. A length of the fetal rat is measured in a sagittal plane and recorded. A complete morphology of the placenta at the umbilical cord placental inlet is displayed. A circumference and area of the placenta are outlined and recorded. Subsequently, moving along the left uterine angle the last gestational sac on an upper left side is located. The above observation and measurement operations are repeated. The same procedure is performed for a first gestational sac at a lower right corner of a right uterine angle and a last gestational sac at an upper right corner of a right uterine angle. Four target gestational sacs from each pregnant rat are collected: lower left, upper left, lower right, and upper right. All measurements should be taken three times and an average should be taken.

Preferably, in the photoacoustic imaging module, a guide pad is placed on the abdomen of the pregnant rat, and a 9 MHz photoacoustic ultrasound probe is used to display the cervix of the pregnant rat in the transverse section of the lower abdomen. The probe is rotated and adjusted to a direction along a long axis of a left uterine angle, and a first gestational sac appearing along a lower place of the left uterine angle is displayed. A depth and focus of the probe is adjusted to make a fetal rat and placenta inside a target gestational sac clear. The probe is switched to a photoacoustic mode in the maximum sagittal section of the placenta. A photoacoustic imaging range is adjusted to include the placenta but not exceed its maximum longitudinal diameter. A distribution of photoacoustic signals is observed, their oxygenation status are calculated and recorded. Subsequently, moving along the left uterine angle the last gestational sac on an upper left side is located. The above observation and measurement operations are repeated. The same procedure is performed for a first gestational sac at a lower right corner of a right uterine angle and a last gestational sac at an upper right corner of a right uterine angle. Four target gestational sacs from each pregnant rat are collected: lower left, upper left, lower right, and upper right. All measurements should be taken three times and an average should be taken.

Preferably, in the planar wave imaging module, the cervix of the pregnant rat is displayed in the transverse section of the lower abdomen of the pregnant rat. After the image is clearly displayed, the probe is rotated and adjusted to a direction along a long axis of a left uterine angle, and a first gestational sac appearing along a lower place of the left uterine angle is displayed. A depth and focus of the probe are adjusted to make a fetal rat and placenta inside a target gestational sac clear. The maximum coronal section of the placenta through the umbilical cord placental inlet is displayed. The probe is switched to a plane wave mode, a speed scale is adjusted to 4.5 cm/s, and the gain is set to 50 dB. A fine vascular structure and color signal distribution are observed. After the image is stable, each target placenta is outlined, the area of the placenta in the section and the area of the blood vessels displayed in the plane wave are calculated, the area of the blood vessels in the plane wave is divided by the area of the placenta, and the blood vessel density of the placenta is obtained. Subsequently, moving along the left uterine angle the last gestational sac on an upper left side is located. The above observation and measurement operations are repeated. The same procedure is performed for a first gestational sac at a lower right corner of a right uterine angle and a last gestational sac at an upper right corner of a right uterine angle. Four target gestational sacs from each pregnant rat are collected: lower left, upper left, lower right, and upper right. All measurements should be taken three times and an average should be taken.

Preferably, in the statistical analysis module, the statistical analysis on the data of the grayscale ultrasound module, the photoacoustic imaging module, and the plane wave imaging module is performed.

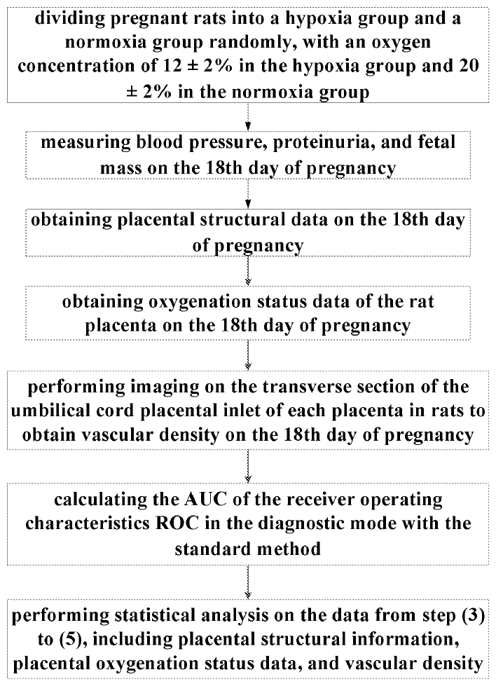

As shown as FIG. 1, a working method for the multimodal imaging means for evaluating tissue oxygenation status is also provided, which comprises the following steps:
(1) dividing pregnant rats into a hypoxia group and a normoxia group randomly, with an oxygen concentration of 12±2% in the hypoxia group and 20±2% in the normoxia group,
(2) measuring blood pressure, proteinuria, and fetal mass on the 18th day of pregnancy,
(3) obtaining placental structural data on the 18th day of pregnancy,
(4) obtaining oxygenation status data of the rat placenta on the 18th day of pregnancy,
(5) performing imaging on the transverse section of the umbilical cord placental inlet of each placenta in rats to obtain vascular density on the 18th day of pregnancy,
(6) calculating the AUC of the receiver operating characteristics ROC in the diagnostic mode with the standard method,
(7) performing statistical analysis on the data from step (3) to (5), including placental structural information, placental oxygenation status data, and vascular density.

It also provides an application of the multimodal imaging means for evaluating tissue oxygenation status, which is used to diagnose preeclampsia and provide a dynamic monitoring in a treatment of preeclampsia.

It is shown that the multimodal imaging means is effective in diagnosing preeclampsia, and the model's AUC reaches 0.82, indicating that the multimodal imaging means has application value in assisting the diagnosis of preeclampsia. A multi-mode imaging means strategy used in an experiment may also provide a new idea for accurate diagnosis of other diseases.

Technologies well-known in the field to which the present invention relates are not described in detail. The descriptions above are only preferred embodiments of the present invention, and are not intended to limit the present invention. Any modifications, equivalent substitutions, improvements and the like made within the spirit and principles of the present invention shall be included in the scope of protection of the present invention.

What is claimed is:

1. A multimodal imaging means for evaluating tissue oxygenation status, comprising:
a test object acquisition module, which is configured to randomly divide pregnant rats into a hypoxia group and a normoxia group, with an oxygen concentration of 12±2% in the hypoxia group and 20±2% in the normoxia group;
a basic data measurement module, which is configured to measure blood pressure, proteinuria, and fetal mass on the 18th day of pregnancy;
a grayscale ultrasound module, which is configured to obtain placental structural information provided by grayscale ultrasound on the 18th day of pregnancy;
a photoacoustic imaging module, which is configured to obtain oxygenation status data of the rat placenta on the 18th day of pregnancy;
a planar wave imaging module, which is configured to perform imaging on a transverse plane of an umbilical cord placental inlet of each placenta in the rats on the 18th day of pregnancy to obtain vascular density;
a calculation module, which is configured to calculate a diagnostic capability AUC of a receiver operating characteristics ROC in a diagnostic mode with a standard method; and
a statistical analysis module, which is configured to perform statistical analysis on data of the grayscale ultrasound module, the photoacoustic imaging module, and the planar wave imaging module.

2. The multimodal imaging means for evaluating tissue oxygenation status according to claim 1, wherein in the grayscale ultrasound module, a 20 MHz probe is used to identify a reproductive system of a pregnant rat, a cervix of the pregnant rat is displayed in a transverse section of a lower abdomen of the pregnant rat, after displaying clearly, the probe is rotated and adjusted to a direction along a long axis of a left uterine angle, and a first gestational sac appearing along a lower place of the left uterine angle is displayed, a depth and focus of the probe are adjusted to make a fetal rat and placenta inside a target gestational sac clear, the fetal rat and placenta are continuously scanned to observe their morphology and positional relationship, a length of the fetal rat is measured in a sagittal plane and recorded, a complete morphology of the placenta at the umbilical cord placental inlet is displayed, a circumference and area of the placenta are outlined and recorded, subsequently, moving along the left uterine angle, locating the last gestational sac on an upper left side, repeating the above observation and measurement operations, performing the same procedure is performed for a first gestational sac at a lower right corner of a right uterine angle and a last gestational sac at an upper right corner of a right uterine angle, collecting four target gestational sacs from each pregnant rat: lower left, upper left, lower right, and upper right, and taking all measurements three times and taking an average.

3. The multimodal imaging means for evaluating tissue oxygenation status according to claim 2, wherein in the photoacoustic imaging module, a guide pad is placed on the abdomen of the pregnant rat, a 9 MHz photoacoustic ultrasound probe is used to display the cervix of the pregnant rat in the transverse section of the lower abdomen, the probe is rotated and adjusted to a direction along a long axis of a left uterine angle, a first gestational sac appearing along a lower place of the left uterine angle is displayed, a depth and focus of the probe is adjusted to make a fetal rat and placenta inside a target gestational sac clear, the probe is switched to a photoacoustic mode in the maximum sagittal section of the placenta, a photoacoustic imaging range is adjusted to include the placenta but not exceed its maximum longitudinal diameter, a distribution of photoacoustic signals is observed, their oxygenation status are calculated and recorded, subsequently, moving along the left uterine angle, locating the last gestational sac on an upper left side, repeating the above observation and measurement operations, performing the same procedure is performed for a first gestational sac at a lower right corner of a right uterine angle and a last gestational sac at an upper right corner of a right uterine angle, collecting four target gestational sacs from each pregnant rat: lower left, upper left, lower right, and upper right, and taking all measurements three times and taking an average.

4. The multimodal imaging means for evaluating tissue oxygenation status according to claim 3, wherein in the planar wave imaging module, the cervix of the pregnant rat is displayed in the transverse section of the lower abdomen of the pregnant rat, after the image is clearly displayed, the probe is rotated and adjusted to a direction along a long axis of a left uterine angle, and a first gestational sac appearing along a lower place of the left uterine angle is displayed, a depth and focus of the probe are adjusted to make a fetal rat and placenta inside a target gestational sac clear, the maximum coronal section of the placenta through the umbilical cord placental inlet is displayed, the probe is switched to a plane wave mode, a speed scale is adjusted to 4.5 cm/s, and the gain is set to 50 dB, a fine vascular structure and color signal distribution are observed, after the image is stable, each target placenta is outlined, the area of the placenta in the section and the area of the blood vessels displayed in the plane wave are calculated, the area of the blood vessels in the plane wave is divided by the area of the placenta, and the blood vessel density of the placenta is obtained, subsequently, moving along the left uterine angle, locating the last gestational sac on an upper left side, repeating the above observation and measurement operations, performing the same procedure is performed for a first gestational sac at a lower right corner of a right uterine angle and a last gestational sac at an upper right corner of a right uterine angle, collecting four target gestational sacs from each pregnant rat: lower left, upper left, lower right, and upper right, and taking all measurements three times and taking an average.

5. The multimodal imaging means for evaluating tissue oxygenation status according to claim 4, wherein in the calculation module, a diagnostic capability AUC of a receiver operating characteristics ROC in a diagnostic mode is calculated with a standard method.

6. The multimodal imaging means for evaluating tissue oxygenation status according to claim 5, wherein in the statistical analysis module, the statistical analysis on the data of the grayscale ultrasound module, the photoacoustic imaging module, and the plane wave imaging module is performed.

7. A working method for the multimodal imaging means for evaluating tissue oxygenation status according to claim 1, comprising the following steps:
(1) dividing pregnant rats into a hypoxia group and a normoxia group randomly, with an oxygen concentration of 12±2% in the hypoxia group and 20±2% in the normoxia group;
(2) measuring blood pressure, proteinuria, and fetal mass on the 18th day of pregnancy;
(3) obtaining placental structural data on the 18th day of pregnancy;
(4) obtaining oxygenation status data of the rat placenta on the 18th day of pregnancy;
(5) performing imaging on the transverse section of the umbilical cord placental inlet of each placenta in rats to obtain vascular density on the 18th day of pregnancy;
(6) calculating the AUC of the receiver operating characteristics ROC in the diagnostic mode with the standard method; and
(7) performing statistical analysis on the data from step (3) to (5), including placental structural information, placental oxygenation status data, and vascular density.

8. An method of using the multimodal imaging means for evaluating tissue oxygenation status according to claim 1, comprising steps of: diagnosing preeclampsia and providing a dynamic monitoring in a treatment of preeclampsia.
